SEQUENCE LISTING (12 sequences)

SEQ ID NO: 1 - DNA, 321 bp, Artificial
  Engineered nucleic acid for FK-506 binding protein domain
ggcgtacaagtagagactataagtcctggtgatggaaggacttttccaaaaagaggacaaacatgtgtagttcattatacgggtatgttggaggacggcaaaaagttcgacagtagtagagatcgtaataaaccattcaaattcatgttgggtaaacaagaagtcattaggggatgggaggagggagtcgctcaaatgtcggttggacaacgtgctaagttaacaatcagccctgactacgcatacggagctacaggacatcctggtattatacctccccacgctaccttggtgtttgacgtcgaactgctgaagttagag

SEQ ID NO: 2 - DNA, 321 bp, Artificial
  Engineered sequence for FK506-binding protein domain
ggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagag

SEQ ID NO: 3 - DNA, 321 bp, Artificial
  Engineered FK506-binding protein domain
ggcgtgcaggtggagaccatcagccccggcgacggcagaaccttccccaagagaggccagacctgcgtggtgcactacaccggaatgctggaggacggcaagaagttcgacagcagcagagacagaaacaagcccttcaagttcatgctgggcaagcaggaggtgatcagaggctgggaggagggcgtggcccagatgagcgtgggccagagagccaagctgaccatcagccccgactacgcctacggagccaccggccaccccggcatcatccccccccacgccaccctggtgttcgacgtggagctgctgaagctggag

SEQ ID NO: 4 - DNA, 321 bp, Homo sapiens
  CDS (1)..(321)
ggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 5 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 6 - DNA, 210 bp, Artificial
  12 copies of zinc finger homeodomain
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcg

SEQ ID NO: 7 - DNA, 121 bp, Artificial
  IL2 minipromoter
aacattttgacacccccataatatttttccagaattaacagtataaattgcatctcttgttcaagagttccctatcactctctttaatcactactcacagtaacctcaactcctgccacaa

SEQ ID NO: 8 - protein, 7 aa, Artificial Sequence
  SV40 nuclear localization signal
PKKKRKV

SEQ ID NO: 9 - protein, 9 aa, Artificial Sequence
  c-myc nuclear localization signal
PAAKRVKLD

SEQ ID NO: 10 - protein, 20 aa, Artificial Sequence
  nucleoplasmin nuclear localization signal
AVKRPAATKKAGQAKKKKLD

SEQ ID NO: 11 - protein, 736 aa, Artificial Sequence
  Adeno-associated virus 9 vp1 capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 12 - protein, 738 aa, Artificial Sequence
  Adeno-associated virus 8 vp1 capsid protein
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

(19) United States
(12) Patent Application Publication
(10) Pub. No.: US 2019/0002915 A1
(43) Pub. Date: Jan. 3, 2019

(54) COMPOSITIONS AND METHODS FOR REGULATABLE ANTIBODY EXPRESSION

(71) Applicant: The Trustees of the University of Pennsylvania, Philadelphia, PA (US)

(72) Inventors: James M. Wilson, Philadelphia, PA (US); Anna P. Tretiakova, Woburn, MA (US); Jenny Agnes Sidrane, Phoenixville, PA (US)

(21) Appl. No.: 16/061,982

(22) PCT Filed: Dec. 14, 2016
(86) PCT No.: PCT/US2016/066487

§ 371 Date: Jun. 13, 2018

Related U.S. Application Data
(60) Provisional application No. 62/267,236, filed on Dec. 14, 2015.

(51) Int. Cl. C12N 15/86; A61K 31/436; A61K 9/00; A61K 9/70; A61K 48/00; C12N 9/90; C07K 14/55; C07K 16/10

(52) U.S. Cl. CPC C12N 15/86; C07K 2317/55; A61K 9/0048; A61K 9/0043; A61K 9/0014; A61K 9/0019; A61K 9/0053; A61K 9/7023; A61K 48/0075; A61K 48/0083; A61K 48/0066; C12N 9/90; C12Y 502/01008; C07K 14/55; C07K 16/1045; C12N 2750/14143; C12N 2750/14171; C12N 2750/14132; C07K 2319/81; C07K 2319/715; A61K 31/436

(57) ABSTRACT

Compositions containing multiple different AAV stock are provided which allow for regulated expression of an immunoglobulin in a variety of tissues. Also provided is a method for regulating the dose of a pharmacologically active immunoglobulin. The method involves co-administering: (a) a first stock of recombinant AAV containing: an activation domain operably linked to expression control sequences comprising a promoter and a first nuclear localization signal; and a DNA binding domain comprising a zinc finger homeodomain and two or more FK506 binding protein domain (FKBP) subunit genes, wherein a first FKBP subunit gene and a second FKBP subunit gene have coding sequences which are no more than about 85% identical to each other, said DNA binding domain being operably linked to a second nuclear localization signal; and (b) a second stock of recombinant AAV comprising at least 2 to about 12 copies of a zinc finger homeodomain which are specific binding partners for the zinc finger homeodomain of the DNA binding domain, and further comprising at least one immunoglobulin expression cassette operably linked to inducible expression control sequences, such that when an effective amount of a rapamycin or rapalog is delivered transcription and expression of the immunoglobulin gene is induced.

### INCORPORATION-BY-REFERENCE OF ELECTRONIC MATERIAL

Applicant hereby incorporates by reference the Sequence Listing being filed electronically herewith under file number “16-7727PCT_ST.25”.

### BACKGROUND OF THE INVENTION

One of the major challenges gene therapy applications face clinically is the ability to control the level of expression or silencing of therapeutic genes in order to provide a balance between therapeutic efficacy and nonspecific toxicity due to overexpression of therapeutic protein or RNA interference-based sequences. Thus, the ability to regulate gene expression is essential as it reduces the likelihood of potentially initiating adverse events in patients. Although genes may be regulated at either the translational or post-transcriptional level, gene regulation at the transcriptional level may offer the greatest safety. There are two classes of gene regulation systems—exogenously controlled gene regulation systems, which rely on an external factor (usually the administration of a drug) to turn transgene expression on or off, and endogenously controlled gene expression systems that rely on physiological stimuli to control transgene expression.

Regulated adeno-associated virus (AAV) vectors are expected to have broad utility in gene therapy, and to date, several regulation systems have exhibited a capability to control gene expression from viral vectors over two orders of magnitude. A variety of expression systems have been developed, including regulated expression systems, which rely on switches triggered by a single drug such as tetracycline, RU486 or ecdysone, or on dimerization triggered by compounds such as a rapalog. One exemplary rapalog, rapamycin, is an orally bioavailable drug and thus finds utility in regulated gene expression in vivo as well as in vitro. Rapalog-regulated gene expression systems are described for example in U.S. Pat. Nos. 6,015,709; 6,117,680; 6,133,456; 6,150,527; 6,187,757; 6,306,649; 6,479,653 and 6,649,595. Two major systems which employ the ARIAD® technology include a system based on homodimerization and a system based on heterodimerization (Rivera et al., 1996, Nature Med, 2(9):1028-1032; Ye et al., 2000, Science 283: 88-91; Rivera et al., PNAS, Vol. 96(15): 8657-8662, 1999).

As summarized in J. Naidoo and D. Young, Neurology Research International, Vol 2012, Article ID 595410, 10 pages, (2011), the rapamycin-regulated gene regulation system relies on the interaction between two transcription factors, one incorporating a DNA-binding domain and the other a DNA activation domain. Each of the transcription factors also contains a heterologous ligand-binding domain that enables their interaction in the presence of the dimerizing drug rapamycin to drive transgene expression. DNA binding is facilitated through the human CMV promoter driven production of a zinc finger homeodomain-1 (ZFHD1) DNA-binding domain fused to three copies of the FK-binding protein (FKBP). Transgene expression is achieved in the presence of rapamycin, which induces dimerization of this DNA-binding protein with a fusion protein consisting of the FKBP-rapamycin-associated protein 1 (FRAP) fused to the NFκB p65 activation domain. Due to the size of this system, two viral vectors are required for delivery of all the components. A 1:1 ratio of transcription factor vector to transgene vector has been described as being sufficient for high induction and low basal transgene expression [L M Sanftner et al, Molecular Therapy, 13(1): 167-174 (2006)]. This system has many of the properties required for use clinically. It is characterized by a high induction ratio, low basal expression, and is composed entirely of human proteins. Additionally, rapamycin can be administered orally and has a pharmacokinetic profile that has been widely studied. The primary issue with this system was that rapamycin functions as an immunosuppressant through blocking FRAP activity [E J Brown et al, Nature, 369 (6483): 756-758 (1994)] and inhibiting progression through the cell cycle at concentrations required for gene regulation. Rapamycin analogs (“rapalogs”) have since been engineered by adding substituents which prevent binding to FRAP while binding to FRAP_{L} mutant domain [J H Bayle et al, Chemistry & biology, 13(1): 99-107 (2006)].

Recombinant AAV vectors (rAAV) have been previously used to express single chain and full length antibodies in vivo. Due to the limited transgene packaging capacity of AAV, it has been a technical challenge to have a tightly regulated system to express heavy and light chains of an antibody using a single AAV vector in order to generate full length antibodies

There remains a need in the art for regulatable systems which can mediate controlled doses of antibodies to a variety of tissues.

### SUMMARY OF THE INVENTION

In one aspect, an AAV composition for regulated expression of a recombinant immunoglobulin is provided. The composition contains at least two different stock of AAV for co-administration. A first stock of recombinant AAV contains a vector genome comprising: (i) an activation domain operably linked to expression control sequences comprising a promoter and a first nuclear localization signal; (ii) an optional linker; and (iii) a DNA binding domain comprising a zinc finger homeodomain and two or more FK506 binding protein domain (FKBP) subunit genes, wherein a first FKBP subunit gene and a second FKBP subunit gene have coding sequences which are no more than about 85% identical to each other, said DNA binding domain being operably linked to a second nuclear localization signal. A second stock of recombinant AAV in which the recombinant AAV contain a vector genome comprising two to twelve copies of a zinc finger homeodomain and a minimal promoter, said homeodomain being specific binding partners for the zinc finger homeodomain of the DNA binding domain (a)(iii), and further comprising at least one expression cassette which comprises at least one immunoglobulin gene operably linked to expression control sequences. Optionally, the second rAAV stock may contain separate expression cassettes for each antibody chain, in which each antibody chain has two to twelve copies of a zinc finger homeodomain and a minimal promoter. Each minimal promoter may be the same or different. The presence of an effective amount of a rapamycin or rapalog induces transcription and expression of the immunoglobulin gene in a host cell co-transfected with the first and second stock.

In another aspect, a method for regulating the dose of a pharmacologically active immunoglobulin is provided. The method comprises co-administering: (a) a first stock of recombinant AAV in which the recombinant AAV contain a vector genome comprising: (i) an activation domain operably linked to expression control sequences comprising a promoter and a first nuclear localization signal; (ii) an optional linker; (iii) a DNA binding domain comprising a zinc finger homeodomain and two or more FK506 binding protein domain (FKBP) subunit genes, wherein a first FKBP subunit gene and a second FKBP subunit gene have coding sequences which are no more than about 85% identical to each other, said DNA binding domain being operably linked to a second nuclear localization signal; and (b) a second stock of recombinant AAV stock in which the recombinant AAV contain a vector genome comprising two to twelve copies of a zinc finger homeodomain, said homeodomain being specific binding partners for the zinc finger homeodomain of the DNA binding domain (a)(iii), and further comprising at least one expression cassette which comprises at least one immunoglobulin gene operably linked to expression control sequences. Further, at the same time as, or following co-administration, a predetermined dose of an inducing agent (e.g. rapamycin or a rapalog) is delivered to induce transcription and expression of the immunoglobulin gene in a host cell co-transfected with the first and second stock. In one embodiment, the inducing agent is delivered at about 1 day following administration of the multi-AAV vector composition.

Still other advantages of the present invention will be apparent from the detailed description of the invention.

### BRIEF DESCRIPTION OF THE DRAWINGS

FIGS. 1A-1F illustrate delivery and regulatable expression following intranasal delivery of a rAAV9-mediated gene in mice. FIGS. 1A-1E illustrate ffLuc expression at day 0 (FIG. 1A), 1 (FIG. 1B), 2 (FIG. 1C), day 3 (FIG. 1D), and day 7 (FIG. 1E). These figures illustrate that ffLuc expression levels within 24 hrs the level of ffLuc expression following either the IN induction or IP induction reached maximal levels and remained stable till 48 hrs at which time expression began to wane and reached background levels within 7 days. A dual rAAV composition was delivered intranasally followed by either topical delivery of a 10 ng/kg rapamycin solution (dotted line FIG. 1F) or intraperitoneal injection of 1 mg/mL rapamycin solution (solid line FIG. 1F). The highest level of induction achieved by the IN or IP delivery of rapamycin (FIG. 1F) was similar to the levels of ffLuc expression conferred by the constitutively expressed AAV2/9.CMV.ffLuc vector (FIG. 1F, black solid line).

FIG. 2 illustrates subretinal injection of AAV8 regulatable dual antibody system in a mouse model results in regulatable expression.

FIG. 3 illustrates inducible antibody expression in the non-human primate eyes. A male rhesus monkey was injected IV with rapamycin at various time points, with OS (line with circle) referring to left eye and OD (line with square) referring to right eye. Antibody expression was measured by ELISA. Several inductions were performed over 4 years. The peak of antibody expression was found to be 14 days post induction and waned to background undetectable levels within 3 months of induction. Over time a decrease of the maximal expression of antibody post-induction was observed (day 0 is day of vector injection).

FIG. 4 provides an alignment of the nucleic acid sequences of FKBP wild-type (wt), and engineered FKBP sequences having about 60% identity (FKBP60) [SEQ ID NO: 1], about 70% identity (FKBP70) [SEQ ID NO: 2], and about 80% identity (FKBPco) [SEQ ID NO: 3] to the wild-type FKBP [SEQ ID NO: 4] sequence. Asterisks (*) below the sequence illustrate conserved bases among the aligned sequences.

FIG. 5 illustrates regulatable expression of constructs containing engineered regulated promoters, pAR1, pAR2, pAR3, and pAR5 and reporter constructs containing firefly luciferase (ffLuc), following induction with either rapamycin (lighter gray bars) or a rapalog AP22594 (28-epi-rapamycin)(darker gray bars) at 0, 0.7812, 1.6, 3.125, 6.25, 12.5, 25 or 30 μg.

FIG. 6 illustrates expression of transgene expression (μg/mL of serum) by all tested regulatable promoter systems when the plasmids containing the modified FKBP subunits were co-transfected with a Z12i-antibody construct instead of ffLuc reporter, following induction with 0 nM, 10 nM, or 1 μM AP22594 rapalog.

FIG. 7A illustrates packaging of the cis-plasmids with the FKBP variant sequences into AAV8 or AAV9 capsids. FIG. 7B provides a linear representation of the vector genome.

FIG. 8 illustrates induction of antibody expression from AAV8 vectors containing Z12i-201IA (an immunoadhesin construct) and the vector carrying the transcription factor with the variant FKBP sequences described herein.

FIG. 9 illustrates induction of ffLUC following intranasal delivery of AAV9.ffLUC using the variant FKBP in the rAAV carrying the transcription factor (Tf) and topical delivery of rapamycin measured at 12, 48 or 120 hours post-induction.

### DETAILED DESCRIPTION OF THE INVENTION

An AAV composition for regulated expression of a recombinant immunoglobulin is provided herein. The system described herein is designed to provide improved safety and the ability to provide for a controlled dose of immunoglobulin in a variety of tissues, and is particularly designed for use in a rapamycin-regulatable expression system. The composition contains a first stock of recombinant AAV containing transcription factor under the control of a suitable promoter and at least a second stock of recombinant AAV containing an antibody under the control of a rapamycin regulatable promoter.

Rapalog-Regulated Expression

In one embodiment illustrated in the examples below, an improved rapamycin/rapalog regulatable system is provided herein. As provided herein, the dose of the immunoglobulin delivered via the AAV vectors provided herein is regulated (controlled) by the regulating or inducing agent (small molecule) delivered to the subject. Thus, the delivery of the rapamycin or rapalog brings together the two intracellular molecules co-delivered via the AAV composition provided herein, each of which is linked to either a transcriptional activator or a DNA binding protein. When these components come together, transcription of the immunoglobulin is activated.

In this system, the dimerizer inducible gene regulation system is comprised of 3 individual components: the activation domain, DNA binding domain, and the inducible promoter upstream of the antibody expression cassette of interest. Typically, the activation domain and DNA binding domain are located on a first rAAV stock and at least a second rAAV stock contains the regulatable promoter and antibody expression cassette(s). In one exemplary embodiment, the activation domain is a fusion of the carboxy terminal from the p65 subunit of NF-kappa B and the large PI3K homolog FRAP domain (FRB), while the DNA binding domain is composed of a zinc finger pair from a transcription factor and a homeodomain joined to two copies of FK506 binding protein (FKBP). As described herein, alternative activation domains may be substituted in the rAAV stock provided herein. In the presence of an inducing agent, e.g., a rapalog such as rapamycin, the DNA binding domain and activation domain are dimerized through interaction of their FKBP and FRB domains, leading to transcription activation of the immunoglobulin.

In one embodiment, two or more different rAAV stock are co-administered. The first rAAV stock contains the transcription factor (tf) and at least a second rAAV contains the immunoglobulin under control of the regulatable promoter. Optionally, two or more rAAV stock may contain the different immunoglobulins for co-delivery with the transcription factor.

More particularly, the first stock of rAAV particles having packaged therein a vector genome containing, at a minimum, sequences encoding an activation domain operably linked to expression control sequences comprising a promoter, intron, Kozak and a first nuclear localization signal and sequences encoding a DNA binding domain comprising a zinc finger homeodomain and two or more FK506 binding protein domain (FKBP) subunit genes, said DNA binding domain being operably linked to a second nuclear localization signal. A second rAAV stock is composed of rAAV particles having packaged therein a vector genome comprising at least 2 to about 12 copies of a zinc finger homeodomain which is a specific binding partner(s) for the zinc finger homeodomain of the DNA binding domain the first rAAV stock. The vector genome of the second rAAV stock also comprises a mini-promoter and at least one expression cassette which comprises at least one immunoglobulin gene operably linked to the rapamycin regulated expression control sequences. In the presence of an effective amount of a rapamycin or rapalog, transcription of the immunoglobulin gene is induced in a regulatable manner.

The rAAV of the first AAV stock containing the transcription factor is designed to have at least one, two, and optionally, three copies of the FKBP sequence. These are termed herein FKBP subunits. Suitably, the subunits are designed to express the same protein, but to have nucleic acids which are divergent from one another in order to minimize recombination. Examples of suitable FKBP sequences are provided herein. In one embodiment, the selected FKBP subunit are less than about 85% identical to each other, i.e., at least about 15% divergent.

Examples of suitable FKBP subunit sequences are provided herein in Example 1 below, in certain embodiments, the FKBP subunits are about 60% to about 80% identical to the wild-type FKBP coding sequence. However, other suitable sequences may be designed.

| | FKBP60 | FKBP70 | FKBPco | FKBPwt |
|---|---|---|---|---|
| FKBP60 | | 79 | 75 | 61 |
| FKBP70 | | | 82 | 74 |
| FKBPco | | | | 81 |
| FKBPwt | | | | |

Optionally, one of the subunit sequences may be a wild-type FKBP sequence. In one embodiment, the wild-type FKBP sequence is located upstream of an engineered FKBP subunit sequence. In another embodiment, the wild-type FKBP sequence is located downstream of an engineered FKBP subunit sequence. In still another embodiment, the wild-type FKBP sequence is sandwiched between two different engineered FKBP subunit sequences. In another embodiment, the wild-type FKBP subunit sequence is not used in the composition of the invention.

The first rAAV stock further comprise an activation domain, which is preferably located upstream of the DNA binding domain. In one embodiment, the activation domain is an activation domain previously described for use in rapamycin-regulatable systems. For example, an activation domain may be a FRAP or FRAP_{L} domain fused to a carboxy terminal from the p65 subunit of NF-kappa B. However, other activation domains may be substituted therefore and used in a composition of the invention. Such other activation domains may include, e.g., VP16, p53, E2F1, or B42.

VP16 refers to the transcriptional activation domain of herpesvirus protein VP16, which is in the carboxy-terminal 78 amino acids and interacts with multiple transcriptional components. See, e.g., Hall and Struhl, J Biol Chem, 277: 46043-46050 (Sep. 23, 2002); the p53 activation domain refers to a tandem of nine amino acid domains (residues 43-63) in tumor protein p53 which has been described as being multi-functional [see. e.g, Kaustov et al, Cell Cycle, 2006 Mar. 5 (5): 489-94 (Epub 2006)]; the E2F1 and E1A12S activation domains are described, e.g., Trouche and Kouzarides, Proc Natl Acad Sci USA, 93: 1439-1442 (February 1996); the bacterially derived B42 activation domain is described, e.g., Luciano and Wilson, Proc Natl Acad Sci, 97(20): 10757-10762 (Sep. 26, 2000)].

Suitably, the first rAAV stock carrying the activation domain and the DNA binding/homodimer may be administered in a ratio of about 1:10 to about 10:1 with the second rAAV stock carrying the antibody expression cassette. Although an excess of the first rAAV stock may be desired, such that a ratio of about 5:1 to about 2:1 is desired, in certain circumstances, a ratio of about 1:1 may be used.

In one embodiment, the rAAV stocks to be co-administered are designed to minimize sequence identity at the nucleic acid level. For example, to the extent that the first rAAV stock and the second rAAV stock each contain a linker (e.g., an IRES or F2A sequence) or each contain a nuclear localization signal, these sequences are designed to have divergent coding sequences. Suitable IRES may be obtained from different sources, e.g., a viral source (e.g., EMCV, FMD, VCIP) or from mammalian origin (c-myc, FGF1A). In another example, each nuclear localization signal selected is designed to have nucleic acid sequences which are least about 15% divergent from each other.

Optionally, the nuclear localization signals may encode the same amino acid sequence, but have divergent nucleic acid sequences. Alternatively, the nuclear localization signals encode different amino acid sequences.

In one embodiment, the nuclear localization signals may be monopartite, e.g., as SV40 [PKKKRKV, SEQ ID NO: 8], c-myc: PAAKRVKLD [SEQ ID NO: 9], or bipartite, e.g., as nucleoplasmin: AVKRPAATKKAGQAKKKKLD [SEQ ID NO: 10].

The second rAAV stock contains coding sequence for an immunoglobulin under the control of a regulatable promoter. The immunoglobulin may be in a single expression cassette or in separate expression cassettes. In one embodiment, there is a linker between a first immunoglobulin coding sequence and a second immunoglobulin coding sequence, which linker may be an F2A, an IRES, or a second copy of the regulatable promoter.

In the examples herein, the second rAAV stock contains 12 zinc finger homeodomains followed by the IL-2 mini-promoter. However, the invention encompasses rAAV vectors having from two to about twelve copies of the zinc finger domains. Similarly, while the examples illustrate delivery of a Fab antibody or an immunoadhesin, it will be understood that other antibody constructs may be expressed using the compositions described herein.

As used herein, a “regulatable promoter” is any promoter whose activity is affected by a cis or trans acting factor (e.g., an inducible promoter), such as an external signal or agent).

A “rapamycin” is a macrolide antibiotic produced by Streptomyces hygroscopicus which binds to a FK506-binding protein, FKBP, with high affinity to form a rapamycin:FKBP complex. The rapamycin:FKBP complex binds with high affinity to the large cellular protein, FRAP, to form an FKBP/rapamycin complex with FRAP. Rapamycin acts as a dimerizer or adapter to join FKBP to FRAP. Rapamycin is also known as sirolimus.

As used herein, the term “rapalog” is meant to include structural variants of rapamycin including analogs, homologs, derivatives and other compounds related structurally to rapamycin. Rapalogs are designed to bind to FRAP_{L}, a mutant of FRAP, but not to wild type FRAP. Such structural variants include modifications such as demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. See, e.g., U.S. Pat. Nos. 6,187,757; 5,525,610; 5,310,903 and 5,362,718, expressly incorporated by reference herein. Exemplary rapalogs include, AP22594 (28-epi-rapamycin) which is illustrated in the examples below, and is particularly well suitable because of provides the inducing activity of rapamycin with significantly lower immunosuppressive properties. This compound may be synthesized by mixing sirolimus (rapamycin) with methylenechlorside in the presence of Ti(OiPr)₄. After a 60 minute reaction, crude product is dissolved in methanol and recrystallized from the methanol/water mixture. Typical final yield after purification is about 50%. However, other suitable methods may be used. Still other exemplary rapalogs include, e.g., temsirolimus, everolimus, ABT578, AP23573 and biolimus. AP26113 (Ariad), AP1510 (Amara, J. F., et al., 1997, Proc Natl Acad Sci USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP.

A “rapamycin-regulated promoter” refers to a promoter the activity of which is regulated by the presence or absence of rapamycin. More particularly, control may be more finely regulated than “on” and “off” and the level of transcription may be controlled by the concentrations or doses of rapamycin provided.

The term “immunoglobulin” is used herein to include antibodies, functional fragments thereof, and immunoadhesins. Antibodies may exist in a variety of forms including, for example, polyclonal antibodies, monoclonal antibodies, camelized single domain antibodies, intracellular antibodies (“intrabodies”), recombinant antibodies, multispecific antibody, antibody fragments, such as, Fv, Fab, F(ab)₂, F(ab)₃, Fab′, Fab′-SH, F(ab′)₂, single chain variable fragment antibodies (scFv), tandem/bis-scFv, Fc, pFc′, scFvFc (or scFv-Fc), disulfide Fv (dsfv), bispecific antibodies (bc-scFv) such as BiTE antibodies; camelid antibodies, resurfaced antibodies, humanized antibodies, fully human antibodies, single-domain antibody (sdAb, also known as NANOBODY®), chimeric antibodies, chimeric antibodies comprising at least one human constant region, and the like. “Antibody fragment” refers to at least a portion of the variable region of the immunoglobulin that binds to its target, e.g., the tumor cell.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

The term “exogenous” typically is used to refer to two elements which are not from the same source, i.e., of different bacterial or viral origin.

The term “vector genome” when used in the context of an rAAV viral particle refers to the nucleic acid sequences packaged in the rAAV capsid. Typically, vector genomes for rAAV are about 3.5 kb to about 5.2 kb, more preferably about 3.7 kb to 5 kb, or about 4 kb to about 4.7 kb. A vector genome contains AAV ITR sequences at the 5′ terminus and 3′ terminus of the nucleic acid sequences (e.g., expression cassette) to be packaged into the vector.

As used herein, the term “virus stock” refers a population or plurality of virus having the same characteristics used for medical purposes. An “rAAV stock” may contain an amount of rAAV having the same AAV capsid and vector genomes, which are measured in genome copies. For example, a stock may contain about 1×10⁹ genome copies (GC) to about 5×10¹³ GC (to treat an average subject of 70 kg in body weight). In one example, the vector is present in an amount of about 3×10¹³ GC, but other amounts such as about 1×10⁹ GC, about 5×10⁹ GC, about 1×10¹⁰ GC, about 5×10¹⁰ GC, about 1×10¹¹ GC, about 5×10¹¹ GC, about 1×10¹² GC, about 5×10¹² GC, or about 1.0×10¹³ GC. However, virus stocks may contain higher or lower amounts. As used herein, virus stock concentration may range from about 250 μL to 100 mL volume liquid (suspension formulary), depending upon the route of delivery. For example, volumes at the end of the range, or even lower, may be suitable for intranasal delivery, whereas other routes (e.g., systemic delivery) may use higher volumes.

As used herein, the term “rAAV particle” is a DNAse resistant recombinant adeno-associated virus which has an assembled capsid and a vector genome packaged into the AAV capsid. An AAV capsid is a self-assembling group of capsid proteins , typically approximately 60 proteins composed of vp1, vp2 and vp3 proteins arranged in an icosahedral symmetry in a ratio of about 1 (vp1): about 1 (vp2): about 10-20 (vp3), depending upon the selected AAV. As used herein, an “expression cassette” refers to a nucleic acid molecule which comprises a gene operably linked to regulatory control elements which direct its transcription and/or expression in a cell. In one embodiment, an expression cassette comprising an immunoglobulin gene(s) (e.g., an immunoglobulin variable region, an immunoglobulin constant region, a full-length light chain, a full-length heavy chain or another fragment of an immunoglobulin construct), promoter, and may include other regulatory sequences therefor, which cassette may be delivered via a genetic element (e.g., a plasmid) to a packaging host cell and packaged into the capsid of a viral vector (e.g., a viral particle). Typically, such an expression cassette for generating a viral vector contains the immunoglobulin sequences described herein flanked by packaging signals of the viral genome and other expression control sequences such as those described herein. In other embodiments, the expression cassette comprises the zinc finger homeodomains, or other gene products, to be expressed. Still other expression cassettes may include other gene products which are expressed or co-expressed with the immunoglobulin regions.

As described above, the term “about” when used to modify a numerical value means a variation of ±10%, unless otherwise specified.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

An “anti-pathogen construct” is a protein, peptide, or other molecule encoded by a nucleic acid sequence carried on a viral vector as described herein, which is capable of providing passive immunity against the selected pathogenic agent or a cross-reactive strain of the pathogenic agent. In one embodiment, the anti-pathogen construct is a neutralizing antibody against the pathogenic agent, e.g., a virus, bacterium, fungus, or a pathogenic toxin of said agent (e.g., anthrax toxin).

A “neutralizing antibody” is an antibody or neutralizing immunoglobulin molecule as defined herein which defends a cell from an antigen or infectious body by inhibiting or neutralizing its biological effect. In one embodiment, “neutralizes” and grammatical variations thereof, refer to an activity of an antibody that prevents entry or translocation of the pathogen into the cytoplasm of a cell susceptible to infection.

An “immunoglobulin molecule” is a protein containing the immunologically-active portions of an immunoglobulin heavy chain and immunoglobulin light chain covalently coupled together and capable of specifically combining with antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein.

An “immunoglobulin heavy chain” is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain.

An “immunoglobulin light chain” is a polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

An “immunoadhesin” is a chimeric, antibody-like molecule that combines the functional domain of a binding protein, usually a receptor, ligand, scFv, variable heavy or light chains, or cell-adhesion molecule, with immunoglobulin constant domains, usually including the hinge and Fc regions.

A “fragment antigen-binding” (Fab) fragment” is a region on an antibody that binds to antigens. It is composed of one constant and one variable domain of each of the heavy and the light chain.

An AAV vector as described herein can comprise one or more nucleic acid sequences, each of which encodes one or more of the heavy and/or light chain polypeptides, or other polypeptides, of an immunoglobulin construct. Suitably, a composition contains one or more AAV vectors which contain all of the polypeptides which form an active immunoglobulin construct in vivo. For example, a full-length antibody consists of four polypeptides: two identical copies of a heavy (H) chain polypeptide and two copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. In this respect, an AAV vector as described herein can comprise a single nucleic acid sequence that encodes the two heavy chain polypeptides (e.g., constant variable) and the two light chain polypeptides of an immunoglobulin construct. Alternatively, the AAV vector can comprise a first expression cassette that encodes at least one heavy chain constant polypeptides and at least one heavy chain variable polypeptide, and a second expression cassette that encodes both light chain polypeptides of an immunoglobulin construct. In yet another embodiment, the AAV vector can comprise a first expression cassette encoding a first heavy chain polypeptide, a second expression cassette encoding a second heavy chain polypeptide, a third expression cassette encoding a first light chain polypeptide, and a fourth expression cassette encoding a second light chain polypeptide.

Typically, an expression cassette for an AAV vector comprises an AAV 5′ inverted terminal repeat (ITR), the immunoglobulin construct coding sequences and any regulatory sequences, and an AAV 3′ ITR. However, other configurations of these elements may be suitable. A shortened version of the 5′ ITR, termed AITR, has been described in which the D-sequence and terminal resolution site (trs) are deleted. In other embodiments, the full-length AAV 5′ and 3′ ITRs are used.

Where a pseudotyped AAV is to be produced, the ITRs in the expression are selected from a source which differs from the AAV source of the capsid. For example, AAV2 ITRs may be selected for use with an AAV capsid having a particular efficiency for targeting CNS or tissues or cells within the CNS. In one embodiment, the ITR sequences from AAV2, or the deleted version thereof (AITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The expression cassette typically contains a promoter sequence as part of the expression control sequences, e.g., located between the selected 5′ ITR sequence and the immunoglobulin construct coding sequence. Tissue specific promoters, constitutive promoters, regulatable promoters [see, e.g., WO 2011/126808 and WO 2013/04943], or a promoter responsive to physiologic cues may be used may be utilized in the vectors described herein. In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., CMV enhancer.

These control sequences are “operably linked” to the immunoglobulin construct gene sequences. As used herein, the term “operably linked” refers to both expression control sequences that are contiguous with the gene of interest and expression control sequences that act in trans or at a distance to control the gene of interest.

In one embodiment, a self-complementary AAV is provided. This viral vector may contain a 45′ ITR and an AAV 3′ ITR. In another embodiment, a single-stranded AAV viral vector is provided. Methods for generating and isolating AAV viral vectors suitable for delivery to a subject are known in the art. See, e.g., U.S. Pat. No. 7,790,449; U.S. Pat. No. 7,282,199; WO 2003/042397; WO 2005/033321, WO 2006/110689; and U.S. Pat. No. 7,588,772 B2]. In one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following US patents, the contents of each of which is incorporated herein by reference in its entirety: U.S. Pat. No. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

A number of suitable purification methods may be selected. Examples of suitable purification methods are described, e.g., in U.S. Patent Applications No. 62/266,351 (AAV1); 62/266,341 (AAV8); 62/266,347 (AAVrh10); and 62/266,357 (AAV9), which are incorporated by reference herein.

The TF expression cassette described herein may contain at least one internal ribosome binding site, i.e., an IRES, located between the coding regions of the heavy and light chains. Alternatively the heavy and light chain may be separated by a furin-2a self-cleaving peptide linker [see, e.g., Radcliffe and Mitrophanous, Gene Therapy (2004), 11, 1673-1674. The expression cassette may contain at least one enhancer, i.e., CMV enhancer. Still other enhancer elements may include, e.g., an apolipoprotein enhancer, a zebrafish enhancer, a GFAP enhancer element, and brain specific enhancers such as described in WO 2013/1555222, woodchuck post hepatitis post-transcriptional regulatory element. Additionally, or alternatively, other, e.g., the hybrid human cytomegalovirus (HCMV)-immediate early (IE)-PDGR promoter or other promoter-enhancer elements may be selected. To enhance expression the other elements can be introns (like Promega intron or similar chimeric chicken globin-human immunoglobulin intron).

The available space for packaging may be conserved by combining more than one transcription unit into a single expression cassette, thus reducing the amount of required regulatory sequences. For example, a single promoter may direct expression of a single cDNA or RNA that encodes two or three or more genes, and translation of the downstream genes are driven by IRES sequences. In another example, a single promoter may direct expression of a cDNA or RNA that contains, in a single open reading frame (ORF), two or three or more genes separated from one another by sequences encoding a self-cleavage peptide (e.g., 2A) and/or a protease recognition site (e.g., furin). The ORF thus encodes a single polyprotein, which, either during or after translation, is cleaved into the individual proteins (such as, e.g., heavy chain and light chain). It should be noted, however, that although these IRES and polyprotein systems can be used to save AAV packaging space, they can only be used for expression of components that can be driven by the same promoter. In another alternative, the transgene capacity of AAV can be increased by providing AAV ITRs of two genomes that can anneal to form head to tail concatamers.

In the examples below, recombinant AAV8 and AAV9 vectors are described. AAV9 vectors are described, e.g., in U.S. Pat. No. 7,906,111, which is incorporated herein by reference. However, other sources of AAV capsids and other viral elements may be selected, as may other immunoglobulin constructs and other vector elements. Methods of generating AAV vectors have been described extensively in the literature and patent documents, including, e.g., WO 2003/042397; WO 2005/033321, WO 2006/110689; U.S. Pat. No. 7,588,772 B2. Suitable AAV may include, e.g, AAV9 [U.S. Pat. No. 7,906,111; US 2011-0236353-A1], rh10 [WO 2003/042397] and/or hu37 [see, e.g., U.S. Pat. No. 7,906,111; US 2011-0236353-A1]. However, other AAV, including, e.g., AAV1, AAV2, AAV3B, AAV4, AAV5, AAV6, AAV7, AAV8 [U.S. Pat. No. 7,790,449; U.S. Pat. No. 7,282,199] and others such as, e.g., those described in a word seems to be missing here may be selected for preparing the AAV vectors described herein.

### Uses and Regimens

Suitably, the compositions are designed to co-administer at least two different AAV vectors carry the nucleic acid expression cassettes encoding the immunoglobulin constructs and regulatory sequences which direct expression of the immunoglobulin thereof in the selected cell.

Following co-administration of the vectors, the inducing agent is used to induce expression of the immunoglobulin constructs in vivo. In one embodiment, antibody expression levels may be controlled in a dose-dependent manner by the dose of inducing agent administered to provide a controlled dosage of antibody.

The use of compositions described herein in therapeutic methods are described, as are uses of these compositions in therapeutic and/or anti-neoplastic regimens, which may optionally involve delivery of one or more other active agents.

As stated above, a composition may contain two or more different AAV vectors apart from the rAAV carrying the transcription factor (rAAV.Tf), each of which has packaged therein different expression cassettes. For example, the two or more different AAV may have different expression cassettes which express immunoglobulin polypeptides which assemble in vivo to form a single active immunoglobulin construct following dosing with the inducing agent. In another example, the two or more AAV may have different expression cassettes which express immunoglobulin polypeptides for different targets, e.g., which provide for two active immunoglobulin constructs.

The compositions can be formulated in dosage units to contain the two or more rAAV, such that each vector stock is present in an amount about 1×10⁹ genome copies (GC) to about 5×10¹³ GC (to treat an average subject of 70 kg in body weight). In one example, the vector concentration is about 3×10¹³ GC, but other amounts such as about 1×10⁹ GC, about 5×10⁹ GC, about 1×10¹⁰ GC, about 5×10¹⁰ GC, about 1×10¹¹ GC, about 5×10¹¹ GC, about 1×10¹² GC, about 5×10¹² GC, or about 1.0×10¹³ GC. Optionally, the rAAV.Tf is present in excess of the rAAV stock with the immunoglobulin expression cassette, e.g., about 10:1 to 1.5:1, or about 5:1 to about 3:1, or about 2:1. However, the ratio of first rAAV stock with the transcription factor to rAAV stock with the immunoglobulin may be about 1:1. In certain embodiments, there may be an excess of rAAV.Ab. Suitable concentrations of these vectors may be readily determined based on the desired volume of liquid suspending agent (e.g., in a range of about 250 μL to 100 mL, or higher or lower, depending upon the route of delivery. For example, volumes at the end of the range, or even lower, may be suitable for intranasal delivery, whereas other routes (e.g., systemic delivery) may use higher volumes.

In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The nuclease resistant particles are then subjected to heat treatment to release the genome from the capsid. The released genomes are then quantitated by real-time PCR using primer/probe sets targeting specific region of the viral genome (usually poly A signal). Another suitable method for determining genome copies are the quantitative-PCR (qPCR), particularly the optimized qPCR or digital droplet PCR [Lock Martin, et al, Human Gene Therapy Methods. April 2014, 25(2): 115-125. doi:10.1089/hgtb.2013.131, published online ahead of editing Dec. 13, 2013].

The rAAV, preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, maltose, and water. The selection of the carrier is not a limitation of the present invention. Optionally, the compositions of the invention may contain, in addition to the rAAV and carrier(s), other conventional pharmaceutical ingredients, such as preservatives, or chemical stabilizers.

Any suitable route of administration for the vector composition may be selected, including, e.g., systemic, intravenous, intraperitoneal, subcutaneous, intrathecal, intraocular (e.g., intravitreal), or intramuscular administration.

The term “dimerizer” refers to a compound (e.g., a small molecule, also termed “pharmacologic agent”) that can bind to dimerizer binding domains of the TF domain fusion proteins and induce dimerization of the fusion proteins. In the constructs described herein, a rapamycin or rapalog is the preferred dimerizer. Any pharmacological agent that dimerizes the domains of the transcription factor, as assayed in vitro can be used. Examples of sutiable rapamycins and its analogs, referred to a “rapalogs” are identified earlier in the specification. Any of the dimerizers described in following can be used: US Publication No. 2002/0173474, US Publication No. 2009/0100535, U.S. Pat. No. 5,834,266, U.S. Pat. No. 7,109,317, U.S. Pat. No. 7,485,441, U.S. Pat. No. 5,830,462, U.S. Pat. No. 5,869,337, U.S. Pat. No. 5,871,753, U.S. Pat. No. 6,011,018, U.S. Pat. No. 6,043,082, U.S. Pat. No. 6,046,047, U.S. Pat. No. 6,063,625, U.S. Pat. No. 6,140,120, U.S. Pat. No. 6,165,787, U.S. Pat. No. 6,972,193, U.S. Pat. No. 6,326,166, U.S. Pat. No. 7,008,780, U.S. Pat. No. 6,133,456, U.S. Pat. No. 6,150,527, U.S. Pat. No. 6,506,379, U.S. Pat. No. 6,258,823, U.S. Pat. No. 6,693,189, U.S. Pat. No. 6,127,521, U.S. Pat. No. 6,150,137, U.S. Pat. No. 6,464,974, U.S. Pat. No. 6,509,152, U.S. Pat. No. 6,015,709, U.S. Pat. No. 6,117,680, U.S. Pat. No. 6,479,653, U.S. Pat. No. 6,187,757, U.S. Pat. No. 6,649,595, U.S. Pat. No. 6,984,635, U.S. Pat. No. 7,067,526, U.S. Pat. No. 7,196,192, U.S. Pat. No. 6,476,200, U.S. Pat. No. 6,492,106, WO 94118347, WO 96/20951, WO 96/06097, WO 97/31898, WO 96/41865, WO 98/02441, WO 95/33052, WO 99/10508, WO 99/10510, WO 99/36553, WO 99/41258, WO 01114387, ARGENT™ Regulated Transcription Retrovirus Kit, Version 2.0 (9109/02), and ARGENT™ Regulated Transcription Plasmid Kit, Version 2.0 (9/09/02), each of which is incorporated herein by reference in its entirety.

In an embodiment, an amount of pharmaceutical composition comprising a dimerizer of the invention is administered that is in the range of about 0.1 to 5 micrograms (μg)/kilogram (kg). To this end, a pharmaceutical composition comprising a dimerizer of the invention is formulated in doses in the range of about 7 mg to about 350 mg to treat to treat an average subject of 70 kg in body weight. The amount of pharmaceutical composition comprising a dimerizer of the invention administered is: 0.1, 0.2, 0.3, 0.4, 0.5, 0.6, 0.7, 0.8, 0.9, 1.0, 1.5, 2.0, 2.5, 3.0, 3.5, 4.0, 4.5 or 5.0 mg/kg. The dose of a dimerizer in a formulation is 7, 8, 9, 10, 20, 25, 30, 35, 40, 45, 50, 55, 60, 65, 70, 75, 80, 85 90, 95, 100,125, 150, 175, 200, 225, 250, 275, 300, 325, 350, 375, 400, 400, 425, 450, 475, 500, 525, 550, 575, 600, 625, 650, 675, 700, 725, or 750 mg (to treat to treat an average subject of 70 kg in body weight). These doses are preferably administered orally. These doses can be given once or repeatedly, such as daily, every other day, weekly, biweekly, or monthly. Preferably, the pharmaceutical compositions are given once weekly for a period of about 4-6 weeks. In some embodiments, a pharmaceutical composition comprising a dimerizer is administered to a subject in one dose, or in two doses, or in three doses, or in four doses, or in five doses, or in six doses or more. The interval between dosages may be determined based the practitioner's determination that there is a need for inhibition of expression of the transgene, for example, in order to ameliorate symptoms caused by expression of the transgene, e.g., toxicity. For example, in some embodiments when the need for transgene ablation is acute, daily dosages of a pharmaceutical composition comprising a dimerizer may be administered. In other embodiments, e.g., when the need for transgene ablation is less acute, or is not acute, weekly dosages of a pharmaceutical composition comprising a dimerizer may be administered.

Pharmaceutical compositions for use as described herein may be formulated in conventional manner using one or more physiologically acceptable carriers or excipients, which may include suspending agents and diluents. The dimerizers and their physiologically acceptable salts and solvates may be formulated for administration by inhalation or insufflation (either through the mouth or the nose) oral, buccal, parenteral, rectal, or transdermal administration. Noninvasive methods of administration are also contemplated.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the dimerizers.

In another embodiment, the rapamycin (rapalog) is delivered via transdermal patch. Such transdermal patch may be applied for 1 day—several months, and time periods in between.

For buccal administration the compositions may take the form of tablets or lozenges formulated in conventional manner.

For administration by inhalation, the dimerizers for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the dimerizers and a suitable powder base such as lactose or starch.

The dimerizers may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The dimerizers may also be formulated in rectal compositions such as suppositories or retention enemas, e.g., containing conventional suppository bases such as cocoa butter or other glycerides.

In addition to the formulations described previously, the dimerizers may also be formulated as a depot preparation. Such long acting formulations may be administered by implantation (for example subcutaneously or intramuscularly) or by intramuscular injection. Thus, for example, the dimerizers may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Also encompassed is the use of adjuvants in combination with or in admixture with the dimerizers of the invention. Adjuvants contemplated include but are not limited to mineral salt adjuvants or mineral salt gel adjuvants, particulate adjuvants, microparticulate adjuvants, mucosal adjuvants, and immunostimulatory adjuvants. Adjuvants can be administered to a subject as a mixture with dimerizers of the invention, or used in combination with the dimerizers of the invention.

In another embodiment, a composition may contain each rAAV stock in an amount of about 1.0×10⁸ genome copies (GC)/kilogram (kg) to about 1.0×10¹⁴ GC/kg, and preferably 1.0×10¹¹ GC/kg to 1.0×10¹³ GC/kg to a human patient. Preferably, each rAAV stock is administered in an amount of about 1.0×10⁸ GC/kg, 5.0×10⁸ GC/kg, 1.0×10⁹ GC/kg, 5.0×10⁹ GC/kg, 1.0×10¹⁰ GC/kg, 5.0×10¹⁰ GC/kg, 1.0×10¹¹ GC/kg, 5.0×10¹¹ GC/kg, or 1.0×10¹² GC/kg, 5.0×10¹² GC/kg, 1.0×10¹³ GC/kg, 5.0×10¹³ GC/kg, 1.0×10¹⁴ GC/kg.

These doses can be given once or repeatedly, such as daily, every other day, weekly, biweekly, or monthly, or until adequate transgene expression is detected in the patient. In an embodiment, replication-defective virus compositions are given once weekly for a period of about 4-6 weeks, and the mode or site of administration is preferably varied with each administration. Repeated injection is most likely required for complete ablation of transgene expression. The same site may be repeated after a gap of one or more injections. Also, split injections may be given. Thus, for example, half the dose may be given in one site and the other half at another site on the same day.

When packaged in two or more viral stocks, the replication-defective virus compositions are preferably administered simultaneously.

In one embodiment, the rAAV compositions may be delivered systemically via the liver by injection, e.g., of a mesenteric tributary of portal vein at a dose of about 3.0×10¹² GC/kg. In another embodiment, the rAAV compositions may be delivered systemically via muscle by up to twenty intramuscular injections, e.g., into either the quadriceps or bicep muscles at a dose of about 5.0×10¹² GC/kg. In another embodiment, the rAAV compositions may be delivered intracranially, e.g., to the basal forebrain region of the brain containing the nucleus basalis of Meynert (NBM) by bilateral, stereotactic injection, at a dose of about 5.0×10¹¹ GC/kg. In another embodiment, the rAAV compositions may be delivered to the CNS intrathecally, by bilateral intraputaminal and/or intranigral injection at a dose in the range of about 1.0×10¹¹ GC/kg to about 5.0×10¹¹ GC/kg. In another embodiment, the rAAV may be delivered to the joints, e.g., by intra-articular injection at a dose of about 1.0×10¹¹ GC/mL of joint volume for the treatment of inflammatory arthritis. In another embodiment, the rAAV may be delivered to the heart, e.g., by intracoronary infusion injection, at a dose in the range of about 1.4×10¹¹ GC/kg to about 3.0×10¹² GC/kg. In another embodiment, the rAAV compositions may be delivered to the retina, e.g., by injection into the subretinal space at a dose of about 1.5×10¹⁰ GC/kg. In view of this information, other means of delivery to these tissues and organs and other doses can be determined by one of skill in the art.

In one aspect, the invention provides a method for regulating the dose of a pharmacologically active immunoglobulin by co-administering at least two different rAAV vector stocks. At least one of the vector stocks provides the transcription factor under the control of a constitutive or tissue-specific promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1 promoter [Invitrogen]. Alternatively, a tissue-specific promoter may be selected. For instance, if expression in skeletal muscle is desired, a promoter active in muscle should be used. These include the promoters from genes encoding skeletal β-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J. Virol., 71:5124-32 (1997); hepatitis B virus core promoter (Sandig et al., Gene Ther., 3:1002-9 (1996)); alpha-fetoprotein (AFP, Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998);

immunoglobulin heavy chain; T cell receptor chain, neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others. Promoters may include a retinal pigmented epithelium (RPE) promoter or a photoreceptor promoter which may be derived from any species. In certain embodiments, the promoter is selected from: human G-protein-coupled receptor protein kinase 1 (GRK1) promoter (Genbank Accession number AY327580); a 292 nt fragment (positions 1793-2087) of the GRK1 promoter (see also, Beltran et al, Gene Therapy 2010 17:1162-74, which is hereby incorporated by reference herein), or a human interphotoreceptor retinoid-binding protein proximal (IRBP) promoter. In certain embodiments, the promoter is the native promoter for the gene to be expressed. In still other embodiments, the promoter is the RPGR proximal promoter (Shu et al, IOVS, May 2012, which is incorporated by reference herein). Other useful promoters include, without limitation, the rod opsin promoter, the red-green opsin promoter, the blue opsin promoter, the cGMP-β-phosphodiesterase promoter, the mouse opsin promoter (Beltran et al 2010 cited above), the rhodopsin promoter (Mussolino et al, Gene Ther, July 2011, 18(7):637-45); the alpha-subunit of cone transducin (Morrissey et al, BMC Dev, Biol, January 2011, 11:3); beta phosphodiesterase (PDE) promoter; the retinitis pigmentosa (RP1) promoter (Nicord et al, J. Gene Med, December 2007, 9(12):1015-23); the NXNL2/NXNL1 promoter (Lambard et al, PLoS One, October 2010, 5(10):e13025), the RPE65 promoter; the retinal degeneration slow/peripherin 2 (Rds/perph2) promoter (Cai et al, Exp Eye Res. 2010 August; 91(2):186-94); and the VMD2 promoter (Kachi et al, Human Gene Therapy, 2009 (20:31-9)). Examples of photoreceptor specific promoters include, without limitation, the rod opsin promoter, the red-green opsin promoter, the blue opsin promoter, the inter photoreceptor binding protein (IRBP) promoter and the cGMP-β-phosphodiesterase promoter.

The other vector stock(s) provide one or more immunoblobulin constructs under the control of an inducible promoter, which inducible promoter is response to a rapamycin or rapalog. More particularly, the physiologically active immunoglobulin may be delivered on a single AAV vector stock. Although less desired, the physiologically active immunoglobulin may be delivered by separate AAV vector stocks, such that the composition contain three (or more) different AAV stocks. Depending upon the target tissue, it may be particularly desired for the AAV stocks to be admixed and delivered simultaneously. For example, this may be particularly desired for intraocular (e.g., intraretinal delivery). In other embodiments, the AAV may be separately formulated and delivered in separate compositions.

Thereafter, the inducing agent (e.g., the selected rapamycin or rapalog) is delivered by any suitable route. In one embodiment, the rapamycin is delivered by a route similar to that by which the rAAV composition was delivered (e.g., intraocular following intraretinal injection of rAAV or intranasal delivery (e.g., topical or spray) following intranasal administration of rAAV, etc. Alternatively, different routes of administration for the rAAV and the rapamycin may be selected. For example, the rapamycin/rapalog may be delivered orally, by injection, intravenously, by transdermal patch, or any other suitable method.

The following examples are illustrative of compositions and methods of the invention.

### EXAMPLES

### Example 1

### Engineered Versions of FKBP (FKBP)

DNA-binding domain fusions (ZFn) containing multiple copies of FKBP were constructed using FKBP coding sequences encoding the wt FKBP sequence and a series of engineered FKBP encoding the same protein, but having divergent nucleic acid sequences. The following tables illustrate the level of identity (%) between the FKBP coding sequences tested.

| | FKBP_60 | FKBP_80 | FKBP_74 | FKBP_CO | FKBP_WT |
|---|---|---|---|---|---|
| FKBP_60 | | 79 | 75 | 75 | 61 |
| FKBP_80 | | | 82 | 82 | 74 |
| FKBP_74 | | | | 93 | 79 |
| FKBP_CO | | | | | 81 |
| FKBP_WT | | | | | |

Cis plasmid constructs having the following elements, from 5′ to 3′ were constructed: promoter, nuclear localization signal (NLS), FRAP_{L} (lipid kinase homolog having rapamycin binding domain), p65 of human NF-κB (transcriptional activation domain), IRES, zinc finger (DNA binding domain), 2 or 3 copies of FK506 binding protein as shown below, and a poly A.

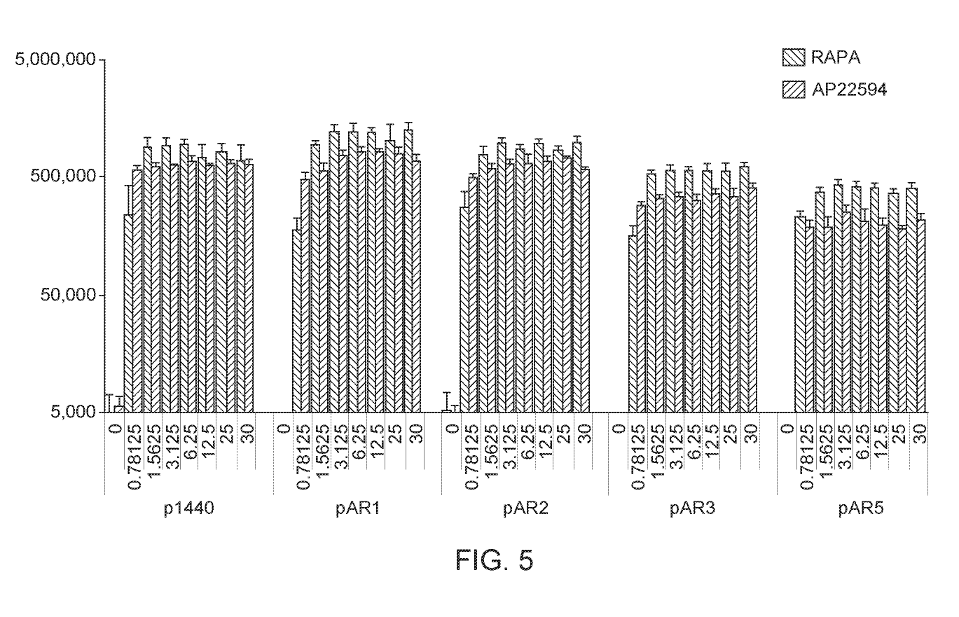

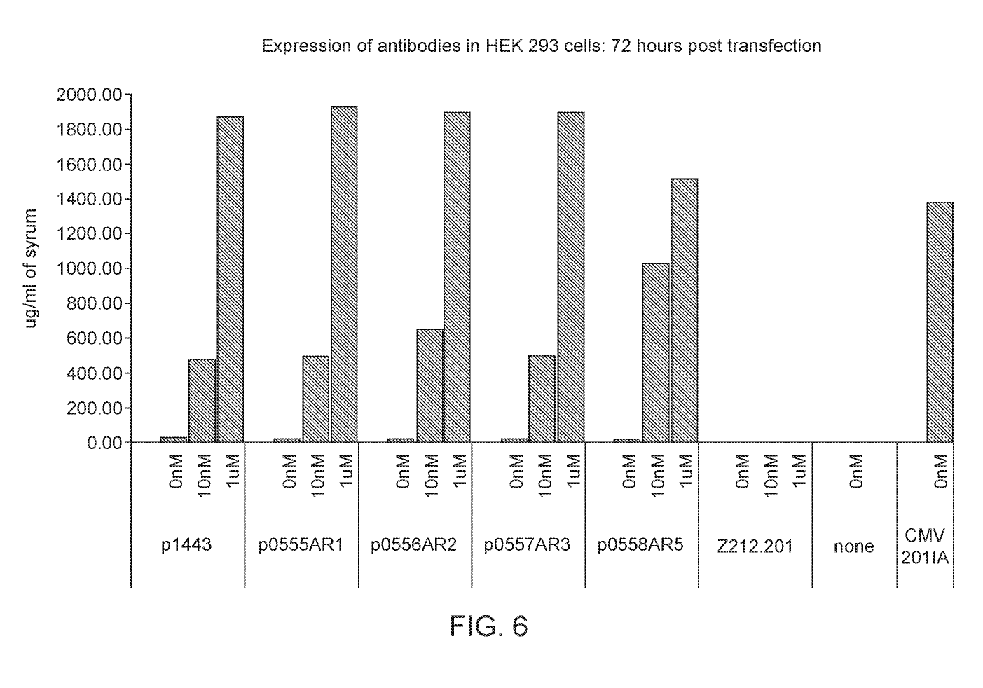

These cis plasmids were tested for in HEK293 cells for the ability to induce expression of ffLuc reporter gene under the control of Z12i promoter, consisting of 12 ZFHD binding sites followed by minimal IL2 promoter. Briefly, HEK293 cells were transfected with cis-plasmids along with the reporter constructs. 24-72 hours post transfection, cells were treated with various concentrations of either rapamycin (sirolimus) or rapalog AP22594 as indicated in FIG. 5. Expression of ffLuc was assessed using dual luciferase detection kit from Promega according to manufacturer's instructions. Treatment with both, rapamycin and rapalog resulted in comparable levels of induction of transgene expression by all TF systems tested. FIG. 6 shows expression of antibody transgene in HEK293 cells, when TF plasmids were co-transfected with Z12i-antibody construct instead of Z12i-ffLuc reporter. Antibodies normally are secreted into supernatant buy the transfected cells. In this experiment, transfection was performed using lipofectamine according to the manufacturer's instructions, and induction was carried out the next day after transfection using indicated amounts of rapalog. At 72 hours post transfection, supernatants were harvested and expression of anti-SIV antibody was assessed using antigen specific ELISA or protein A ELISA. As in case of ffLuc, the performance of all AR variants was better than constitutive expression by CMV-Ab positive control plasmid, with the exception of Ar5, which gave expression comparable to CMV construct: 5′-FKBPco-FKBPwt-FKBP(74 or 60)-3′. As a result five versions were created:

AR1—FKBPco-FKBPwt

AR2—FKBPco-FKBP74

AR3—FKBPco-FKBP60

AR4—FKBPco-FKBPwt-FKBP74 (not tested)

AR5—FKBPco-FKBPwt-FKBP60

### Example 2

### Intranasal Delivery AAV9 Regulatable Dual Antibody System in Mice

A. Materials and Methods

1. Vector Production

AAV2/9.Z12i.ffLuc and AAV2/9.CMV.Tf were used in this example. These vectors were prepared as described in S J Chen et al, Hu Gene Therapy, 24: 270-278 (August 2013). AAV2/9.Z12i.ffLuc contains:

AAV2/9: AAV9 viral particle having an AAV9 capsid [having the amino acid sequence of GenBank accession::AAS99264, reproduced in SEQ ID NO: 11; U.S. Pat. No. 7,906,111 and WO 2005/033321, which are incorporated by reference herein] and a vector genome packaged therein having inverted terminal repeat sequences from AAV2 flanking the expression cassette containing Z12i.ffLuc;

ITR: inverted terminal repeats (ITR) of AAV serotype 2 (168 bp). In one embodiment, the AAV2 ITRs are selected to generate a pseudotyped AAV, i.e., an AAV having a capsid from a different AAV than that the AAV from which the ITRs are derived.

Between the AAV2 ITRs is the Z12i.ffLuc expression cassette: the coding sequence for a reporter gene, firefly luciferase (ffLuc) under control of a ubiquitous, inducible promoter (Z12i);

The Z12i contains 12 copies of the binding site for ZFHD (Z12) (SEQ ID NO: 6)) followed by minimal promoter from the human interleukin-2 (IL-2) gene (SEQ ID NO: 7). This may be induced by rapamycin or certain rapalogs. Variants of this may be used, e.g., which contain from 2 to about 20 copies of the binding site for ZFHD followed by a promoter, e.g., the minimal promoter from IL-2 or another selected promoter.

AAV2/9.CMV.Tf is a recombinant AAV2 viral particle, having an AAV2 capsid with AAV2 ITRs as described above, with the exception that the expression cassette contains transcription factor (tf) coding sequences under the control of the constitutive cytomegalovirus promoter. This vector was prepared as described previously in Auricchio, A., et al. (2002).] for AAV2/2 (also termed AAV-CMV-TF1Nc). In addition to containing the rapamycin-regulated transcription factor, this construct a nuclear localization signal from human c-myc (PAAKRVKLD, SEQ ID NO: 9); a chimeric intron (pCI) downstream of the transcription start site; and 4) the 3 untranslated region is derived from the human growth hormone gene.

2. Vector Injections

C57BL/6 mice (6 to 8 weeks of age) were purchased from Charles River Laboratories (Wilmington, Mass.) and kept under pathogen-free conditions at the Animal Facility of the Translational Research Laboratories. Mice were anesthetized using an intraperitoneal (IP) injection of ketamine/xylazine. For vector administrations, mice were inoculated intranasally (IN) with 12.54 in the right and left nostril for a total dose of 10¹¹ genome copies (GC) in 25 μL. All animal procedures were approved by the Institutional Animal Care and Use Committees of the University of Pennsylvania.

3. Inductions

Topical (IN) application: mice were anaesthetized with ketamine/xylazine and 5 mins later 5 μL of rapamycin solution for a total dose of 10 mg/kg was delivered to the right and left nostril IN. Systemic (IP) application: mice were physically restrained and injected IP with 50 μL of a 1 mg/mL rapamycin solution.

4. Imaging

Mice were anaesthetized with ketamine/xylazine and 5 mins later 10 μL of 15 mg/ml D-luciferin (Caliper, USA) was delivered to the right and left nostril IN. Five mins later mice were imaged using the IVIS® Xenogen imaging system [Perkin-Elmer]. Quantitation of signal was calculated using the Living Image® 3.0 Software.

B. Results

The level of gene expression achieved in the nasal airways following rapamycin induction was examined. Briefly, C57B1/6 mice (n=5/group) were dosed with 10¹¹ GC of both AAV2/9.Z12i.ffLuc and AAV2/9.CMV.Tf in a volume of 25 μL (12.5 μL per nostril). As negative controls, groups of mice were dosed with a) AAV2/9.Z12i.ffLuc alone, b) AAV2/9.CMV.Tf alone and c) PBS mice. As a positive control for the maximal expression of ffLuc, mice (n=5) were injected with AAV2/9.CMV.ffluc. To maximize the safety of the inducible vector system we opted to restrict expression to the nasal epithelium by delivering rapamycin topically (directly to the nose) or systemically (IP).

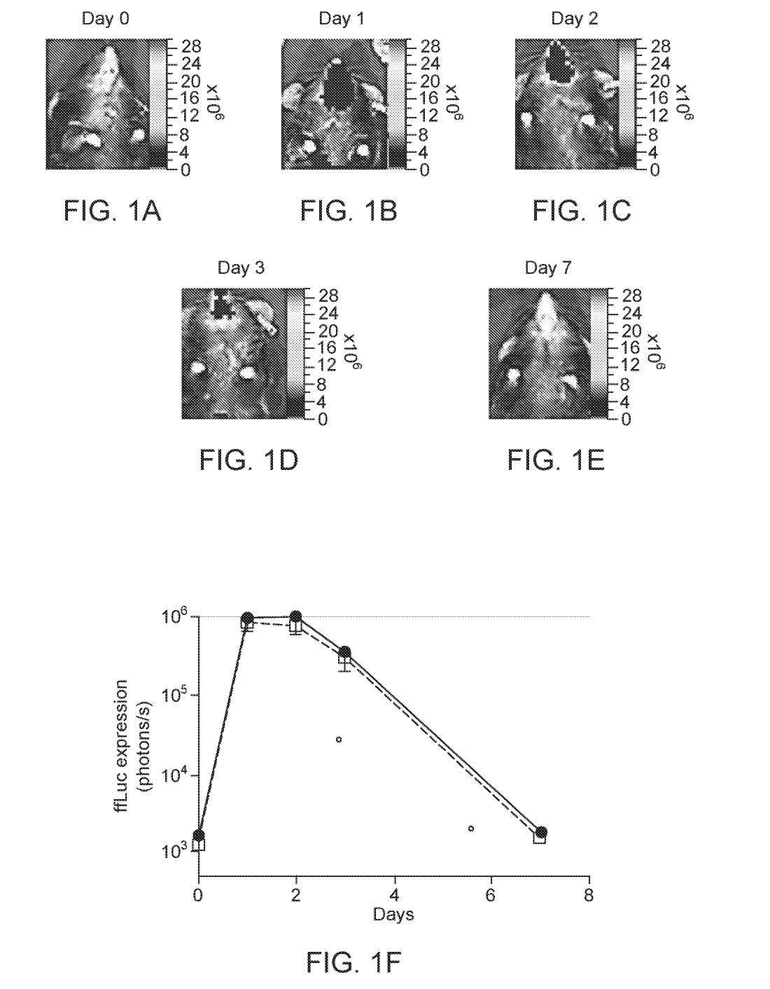

Within 24 hrs the level of ffLuc expression following either the IN induction (FIG. 1A) or IP induction reached maximal levels and remained stable till 48 hrs at which time expression began to wane and reached background levels within 7 days. The delivery of rapamycin to the nose or systemically was well delivered and mice did not exhibit any signs of behavioral distress. More importantly, the onset and kinetics of ffLuc expression was similar when evaluated twenty-eight days later. Mice were given rapamycin IN (FIG. 1B, dotted line) or IP (FIG. 1B, solid line). Interestingly, the maximal level of induction achieved by the IN or IP delivery of rapamycin (FIG. 1B) was similar to the levels of ffLuc expression conferred by the constitutively expressed AAV2/9.CMV.ffLuc vector (FIG. 1B, black solid line).

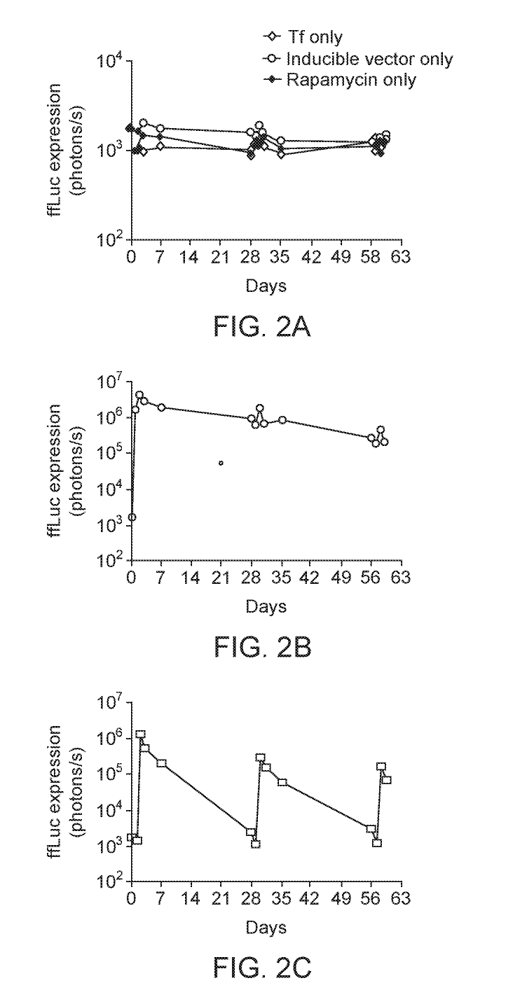

Three separate inductions using rapamycin delivered IN were conducted over three months. See FIG. 2A. Following rapamycin induction, as expected, no luciferase expression was noted when the inducible vector was delivered alone, when the transcription factor plasmid was delivered alone, or when the rapamycin only (no vectors) was injected IN. See FIG. 2B. Interestingly, a non-specific 2-fold increase in ffLuc expression was observed 24 hrs after each one of the three rapamycin inductions of mice given the AAV2/9.CMV.ffLuc vector. See FIG. 2C. Maximal induction was achieved within 24 hrs, was stable to 48 hrs and began to decline at 72hrs to reach background levels by seven days. The kinetics of induction were similar for all three time points (days 28 (noted as day 0), 56 (noted as day 28) and 84 (noted as day 56) post vector administration).

### Example 3

### Subretinal Injection of AAV8 Regulatable Dual Antibody System in Mouse Model

A. Materials and Methods—Mice

1. Vector Production

AAV2/8.CMV.tf was prepared by triple transfection as AAV-CMV-TF1Nc, substituting AAV8 capsid for the AAV9 capsid described in the preceding example. The sequence of the AAV8 vp1 capsid is reproduced in SEQ ID NO: 12.

AAV2/8.Z12.IL2.FabH.FF2A.FabL.BGH was prepared as described in S J Chen et al, Hu Gene Therapy, 24: 270-278 (August 2013), by substituting the firefly luciferase coding sequence with a bicistronic coding sequence containing an antibody heavy chain (FabH), a furin 2A self-cleaving protein, an antibody light chain (FabL), and bovine growth hormone poly A (BGH).

2. Mice

For experiments C57BL/6 were purchased from Charles River Laboratories (Wilmington, Mass., USA) and used at 6-8 weeks of age. Mice were housed under specific pathogen-free conditions at the University of Pennsylvania's Translational Research Laboratories. All animal procedure protocols were approved by the Institutional Animal Care and Use Committee of the University of Pennsylvania.

3. Subretinal Injection in Mouse Eye

AAV8 vector (3×10⁹ genome copies; GC in 2 μL) was delivered using a trans-scleral method in anaesthetized C57BL/6 mice. In brief, the eyelid was opened by blunt separation with forceps and a slight amount of periocular pressure was applied to slightly proptose the eyeball and an incision made into the sclera with a 30½-gauge needle. The underlying sclera was exposed by cutting the conjunctiva with a 30½-gauge needle. The conjunctiva adjacent to the cornea was then grasped and rotated with forceps to allow optimal exposure of the injection site and using a 30½-gauge needle a hole was made in the sclera. The tip of a 33 gauge blunt-tip needle was mounted on a 25 μl Hamilton automatic microinjection syringe Lab Animal Studies Injector (LASI; Hamilton Company, Reno, Nev., USA) and introduced into the incision tangentially to the surface of the globe. The needle was then passed along the inner surface of the sclera choroid with the tip entering approximately 1 mm into the SR space and vector released by activating the plunger via a foot pedal. Following injection of the 2 μl of AAV vector solution, the needle was carefully withdrawn and the conjunctiva repositioned. Topical ointment (PredG, Allergan Pharmaceuticals) was applied to the corneas to minimize drying of the tissue while it healed. No adverse events or mortality were observed during these experiments.

B. Results

Inducible Antibody expression in mouse eye. C57BL/6 mice were injected subretinally (SR) with:
Group 1: 3×10⁹ GC AAV2/8.Z12.IL2.AbH.FF2A.AbL and AAV2/8.CMV.Tf;
Group 2: 3×10⁹ GC of AAV2/8.Z12.IL2.AbH.FF2A.AbL;
Group 3: 3×10⁹ GC of AAV2/8.CMV.Ab;
Group 4: PBS diluent
Group 5: 3×10⁹ GC of AAV2/8.Z12.IL2.AbH.FF2A.AbL and AAV2/8.CMV.Tf (no rapamycin induction).

Mice were induced with 0.5 mg/kg of rapamycin injected IP on either day 14 or day 28 and 24 hrs later eyes removed and homogenates prepared. Antibody expression was measured by ELISA and was observed in group 1 following induction either 14 or 28 days after the single vector delivery but was on average 5-fold lower than that achieved by the constitutive AAV8.CMV.Ab vector.

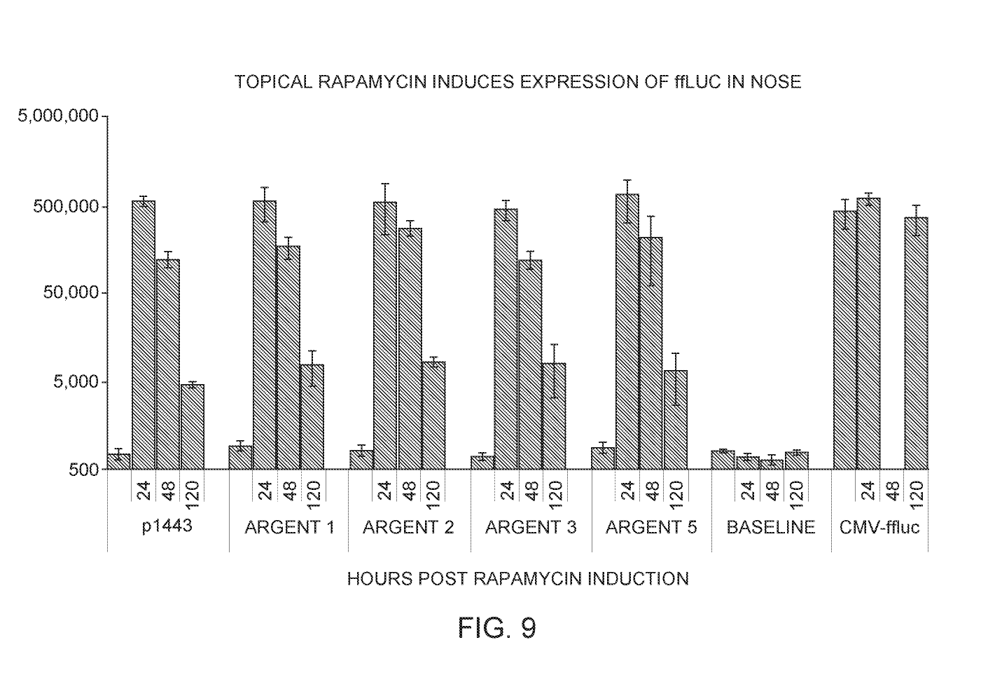

A similar study was performed using the vectors described in Example 1, delivered intranasally to C57B1/6 mice followed by rapamycin induction as described in the preceding sections. FIG. 9 illustrates expression levels observed 24, 48, and 120 hours following topical rapamycin induction.

### Example 4

### Subretinal Injection of AAV8 Regulatable Dual Antibody System in Non-Human Primate (NHP) Model

A. Materials and Methods—NHP

1. NHP

For this experiment a 3 year old male Macaca mulatta (rhesus monkeys) was purchased from Covance Research Products, Inc.

2. Subretinal Injection in NHP Eye

For subretinal (SR) vector injections, a needle was inserted through a sclerotomy 2 mm posterior to the limbus at the 2 or 10 o'clock position. It was then advanced through the vitreous to penetrate the retina in the posterior pole. Under microscopic control, up to 100 μL of anterior chamber fluid was removed and 150 μL of viral vector solution diluted in phosphate buffered saline (PBS) was injected into the subretinal space, thereby raising a dome-shaped retinal detachment (bleb). The detachment covered a small fraction (approximately ⅛th) of the area of the retina in the macula. The NHP received AAV8 vectors injected subretinally into the left (OS) and right (OD) eyes. The two vectors that were injected were: AAV2/8.CMV.TF1Nc (alternate name for AAV2/8.CMV.TF) and AAV2/8.Z12i.AbH.FF2A.AbL.BGH. The vectors were mixed at 1:1 ratio at 10¹¹ GC and delivered SR in a total volume of 150 μL. No adverse events or mortality were observed during these experiments.

3. Induction

Macaques were induced at various times following the SR vector injection. Rapamycin (2 mg/kg) was delivered IV in a solution of 1.2% Tween 80, 27.1% Polyethylene Glycol, 71.7% Nuclease Free Water. At designated times post vector delivery, a tap was performed on the OD eye (baseline) and the NHP then injected IV with 2 mg/kg rapamycin. Twenty four hours later a tap was performed in the OS eye to assess induction of gene expression.

B. Results

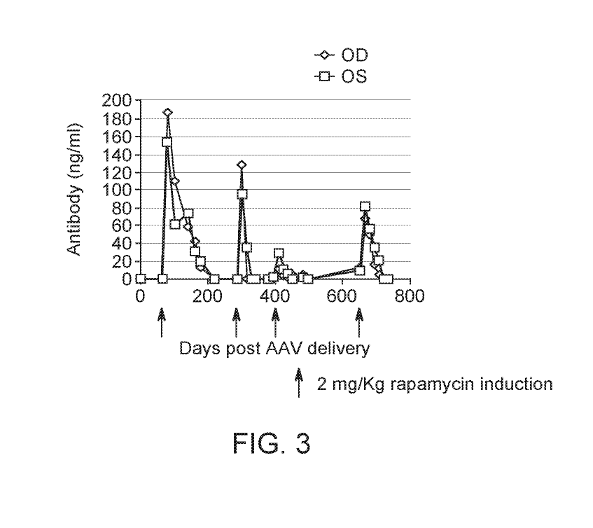

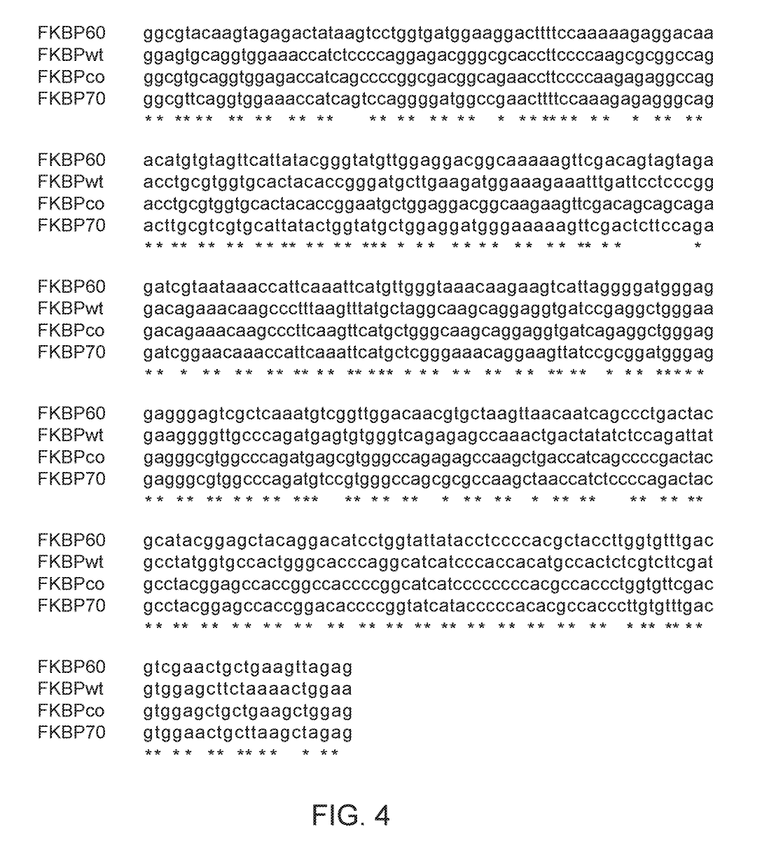

Inducible Antibody expression in the NHP eyes. A male rhesus monkey was injected IV with rapamycin at various time points (FIG. 4). Antibody expression was measured by ELISA. Several inductions were performed over 4 years. The peak of Antibody expression was found to be 14 days post induction and waned to background undetectable levels within 3 months of induction (FIG. 3). Over time a decrease of the maximal expression of Antibody post induction was observed (day 0 is day of vector injection)

### Example 5

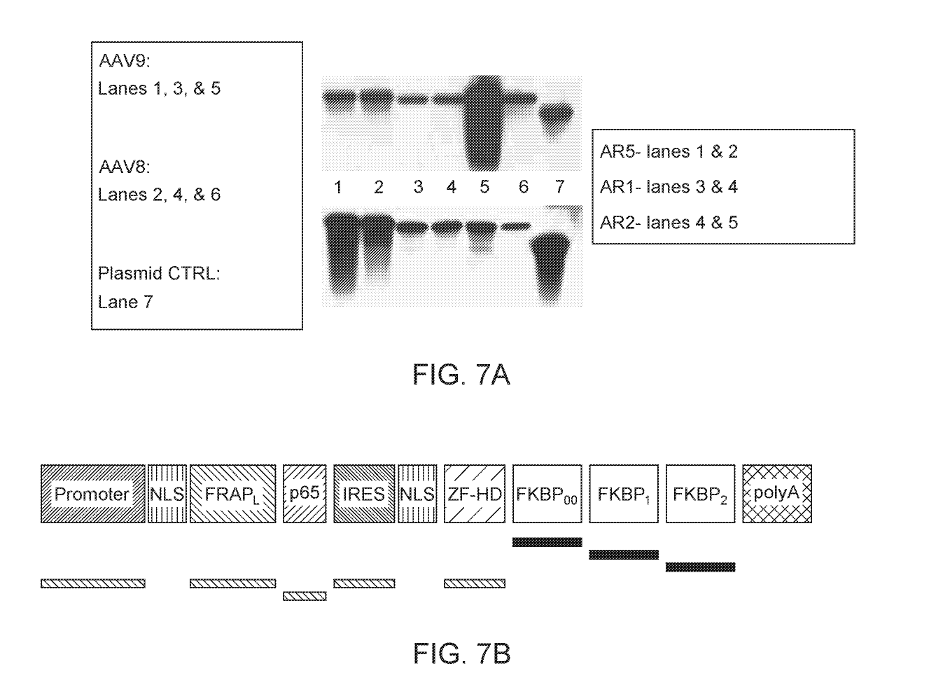

AAV8 and AAV9 capsids were packaged such that they incorporate Ar1, 2 or 5 variants of the TF configurations. Ar1 contains two copies of FKBP- FKBPco and FKBPwt, Ar 2 contains two copies of FKBP- FKBLco followed by FKBP74, and Ar 5 contains 3 copies of FKBP-FKBPco followed by FKBP wt and by FKBP60. DNA was extracted from the packaged capsids, and fractionated using enaturing agarose electophoresis. Fractionation was followed by the transfer to nitrocellulose membrane and Southern Blot hybridization to determine the integrity of the packaged DNA. Top panel shows the results of hybridization of DNA extracted from packager AAV9 capsids, and bottom panel shows the results of southern blot hybridization for the DNA extracted from AAV8 capsids. Lanes 1, 2 and 5 show significant smearing of the DNA, suggesting that the DNA packaged inside the capsids is not stable. Lanes 3, 4 and 6 show no smearing. Lanes 1 & 2 contain DNA extracted from AAV9 and AAV8 capsids that were packaged using Ar5. The data suggest that Ar5 DNA containing 3 copies of FKBP is unstable. Lanes 3 & 4 contain Ar 1 in the context of AAV9 and AAV8 capsids, and the DNA appears stable. Lanes 5 & 6 contain Ar 2 in the context of AAV9 and AAV8 capsids, and is appears that in the context of AAV9 DNA is not stable, but it is stable in the context of AAV8. See, FIG. 7.

### Example 6

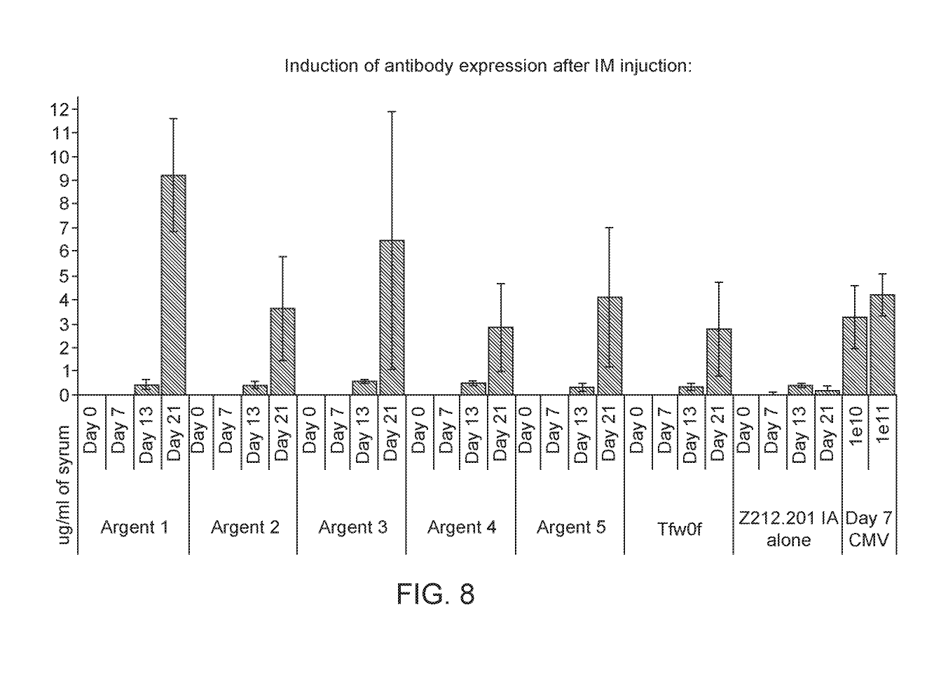

RAG KO mice were purchased from Jackson labs. Z12i-2014A and TF plasmids were packaged into AAV8 vectors using triple transfection method. Vectors were titered using qPCR. Mice were injected IM using 30 ul of mixture of two vectors: first vector is Z12i-2011A at a dose of 5×10¹⁰ GC per mouse, and second vector is one of the TF vectors, AR1-AR5, also at a dose of 5×10¹⁰ GC per mouse. First induction with AP22594 was carried out on day 13 after AAV8 administration. AP22594 was administered IP at a dose of 1 mg/kg. On day 21 post AAV8 administration, orbital bleeds were collected and the level of circulating 2011A antibody was measured by protein A ELISA. The data is presented on FIG. 8. Ar1 shows the highest level of induction, in comparison to the other TF variants. Induced expression peaked at Day 28 (data not shown), started to decline at Day 35, and returned to barely detectable on Day 49, the time frame that is consistent with antibody half-life being 1-2 weeks.

### Sequence Listing Free Text

The following information is provided for sequences containing free text under numeric identifier <223>.

| SEQ ID NO: (containing free | |
|---|---|
| text) | Free text under <223> |
| 1 | <223> Engineered nucleic acid for |
| | FK-506 binding protein domain |
| 2 | <223> Engineered sequence for |
| | FK506-binding protein domain |
| 3 | <223> Engineered FK506-binding |
| | protein domain |
| 6 | <223> 12 copies of zinc finger |
| | homeodomain |
| 7 | <223> IL2 minipromoter |
| 8 | <223> SV40 nuclear localization |
| | signal |
| 9 | <223> c-myc nuclear localization |
| | signal |
| 10 | <223> nucleoplasmin nuclear |
| | localization signal |
| 11 | <223> Adeno-associated virus 9 |
| | vp1 capsid protein |
| 12 | <223> Adeno-associated virus 8 |
| | vp1 capsid protein |

All publications, patents, and patent applications cited in this application, as well as U.S. Provisional Patent Application No. 62/267,236, filed Dec. 14, 2015, are hereby incorporated by reference in their entireties as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims.

1. An AAV composition for regulated expression of a recombinant immunoglobulin, said composition comprising:
(a) a first stock of recombinant AAV in which the recombinant AAV contain a vector genome comprising:
(i) an activation domain operably linked to expression control sequences comprising a promoter and a first nuclear localization signal; and
(ii) a DNA binding domain comprising a zinc finger homeodomain and two or more FK506 binding protein domain (FKBP) subunit genes, wherein a first FKBP subunit gene and a second FKBP subunit gene have coding sequences which are no more than about 85% identical to each other, said DNA binding domain being operably linked to a second nuclear localization signal; and
(b) a second stock of recombinant AAV in which the recombinant AAV contain vector genome comprising at least 2 to about 12 copies of a zinc finger homeodomain, said homeodomain being specific binding partners for the zinc finger homeodomain of the DNA binding domain (a)(iii), and further comprising at least one expression cassette which comprises at least one immunoglobulin gene operably linked to expression control sequences,
wherein the presence of an effective amount of a rapamycin or rapalog induces transcription and expression of the immunoglobulin gene in a host cell co-administered with the first and second stock.

2. The AAV composition according to claim 1, wherein the vector genome further comprises an intron and/or a Kozak sequence upstream of the first nuclear localization signal.

3. The AAV composition according to claim 1, wherein one of the first or second FKBP coding sequence is the wild-type coding sequence.

4. The AAV composition according to claim 1, wherein one of the first or second FKBP is about 60% to about 80% identical to the wild-type FKBP coding sequence.

5. The composition according to claim 1, wherein the AAV composition comprising an activation domain is selected from the group consisting of (a) a FRAP or FRAP_{L} domain fused to a carboxy terminal from the p65 subunit of NF-kappa B; (b) VP16; (c) p53: (d) E2F1, or (e) B42.

6. The composition according to claim 1, wherein the first stock (a) and the second stock (b) are present in a ratio of about 1:10 to about 10:1.

7. The composition according to claim 1, wherein the promoter in the first stock (a) is a constitutive promoter.

8. The composition according to claim 1, wherein the promoter in the first stock (a) is a tissue specific promoter.

9. The composition according to claim 1, wherein the first nuclear localization signal is selected from c-myc or SV4-T-antigen nuclear localization signals.

10. The composition according to claim 1, wherein the second AAV stock is capable of regulatably expressing an immunoglobulin construct selected from a full length antibody, an immunoadhesin, a single chain antibody, or a Fab fragment.

11. The composition according to claim 1, wherein the second AAV stock further comprises a linker between a first immunoglobulin coding sequence and a second immunoglobulin coding sequence, wherein said linker is selected from an F2A sequence, an IRES sequence, or a second inducible promoter.

12. The composition according to claim 11, wherein the linker is an IRES having a sequences selected from the group consisting of: IRES's of viral origin EMCV, FMD, VCIP, and IRES's of mammalian origin CMYC, FGF1A.

13. The composition according to claim 1, wherein the first and the second nuclear localization signal have coding acid sequences which are least about 15% divergent.

14. The composition according to claim 13, wherein the first and second nuclear localization signals encode the same amino acid sequence.

15. The composition according to claim 13, wherein the first and the second nuclear localization signal encode different amino acid sequences.

16. The composition according to claim 1, wherein the second AAV stock comprises up to 12 zinc finger homeodomains.

17. A composition according to claim 1 which is administrable to a subject for regulating the dose of a pharmacologically active immunoglobulin in a regimen further comprising delivering an effective amount of a rapamycin or rapalog to induce transcription and expression of the immunoglobulin gene in a host cell co-transfected with the first and second stock

18. The composition according to according to claim 17, wherein the rapamycin or rapalog is rapamycin.

19. The composition according to claim 17, wherein the recombinant AAV are co-administered intravitreally and the rapamycin is delivered to the eye.

20. The composition according to claim 17, wherein the recombinant AAV are co-administered intranasally and the rapamycin is delivered topically.

21. The composition according to claim 17, wherein the recombinant AAV are co-administered intramuscularly and the rapamycin is delivered orally, topically, via transdermal patch, or subcutaneous injection.

22. The composition according to claim 17, wherein the recombinant AAV are co-administrable intravenously and the rapamycin is deliverable orally, by transdermal patch, or by subcutaneous injection.

23. A method for regulating the dose of a pharmacologically active immunoglobulin, the method comprising co-administering:
(a) a first stock of recombinant AAV in which the recombinant AAV contain a vector genome comprising:
(i) an activation domain operably linked to expression control sequences comprising a promoter and a first nuclear localization signal; and
(ii) a DNA binding domain comprising a zinc finger homeodomain and two or more FK506 binding protein domain (FKBP) subunit genes, wherein a first FKBP subunit gene and a second FKBP subunit gene have coding sequences which are no more than about 85% identical to each other, said DNA binding domain being operably linked to a second nuclear localization signal; and
(b) a second stock of recombinant AAV in which the recombinant AAV contain a vector genome comprising at least 2 to about 12 copies of a zinc finger homeodomain, said homeodomain being specific binding partners for the zinc finger homeodomain of the DNA binding domain (a)(iii), and further comprising at least one expression cassette which comprises at least one immunoglobulin gene operably linked to expression control sequences,
and delivering an effective amount of a rapamycin or rapalog to induce transcription and expression of the immunoglobulin gene in a host cell co-transfected with the first and second stock.
